(19) United States
(12) Patent Application Publication
(10) Pub. No.: US 2008/0033469 A1
(43) Pub. Date: Feb. 7, 2008

(54) BLOOD WITHDRAWAL SYSTEM

(76) Inventors: Sven Winheim, Leimen (DE); Bruno Thoes, Quierschied (DE); Joachim Dopper, Darmstadt (DE)

Correspondence Address: BAKER & DANIELS LLP / ROCHE, 300 NORTH MERIDIAN STREET, SUITE 2700, INDIANAPOLIS, IN, 46204, US

(21) Appl. No.: 11/832,864

(22) Filed: Aug. 2, 2007

(30) Foreign Application Priority Data
Aug. 2, 2006 (EP) EP06016069.4

(51) Int. Cl. A61B 5/151

(52) U.S. Cl. 606/181

(57) ABSTRACT

A blood withdrawal system for producing blood from a body part for diagnostic purposes, comprising a housing with a lancet guide capable of guiding a lancet on a predetermined puncturing path and a lancet drive for driving a puncturing movement of a lancet on the predetermined path. The lancet drive comprises a drive rotor driven by a drive spring and rotates about an axis during the puncturing movement, and a coupling mechanism which converts the rotational movement of the drive rotor into a puncturing movement, wherein the lancet is moved during a forward phase of the puncturing movement in the puncturing direction until its tip penetrates into the body part to create a wound and is retracted from the skin during a retraction phase of the puncturing movement. The coupling mechanism includes a translation element coupled to the lancet and guided by a guide on a movement path.

### BACKGROUND OF THE INVENTION

### RELATED APPLICATION

This application is related to and claims priority to European Application Serial No. 06016069.4, filed Aug. 2, 2006, the disclosure of which is expressly incorporated by reference herein.

1. Field of the Invention

The present invention relates to a blood withdrawal system for producing blood from a body part for diagnostic purposes.

2. Description of the Related Art

To take blood in small amounts for diagnostic purposes from a body part, e.g., from a finger or earlobe, lancets are used; these lancets have a tip which punctures a wound in the corresponding body part. This is done manually by specially trained personnel or by using special blood withdrawal systems including a puncture device and respective lancets.

A lancet drive for driving a puncturing movement of the lancet is provided in the housing of the puncture devices and is formed in many devices as a rotor drive in which the rotation of a drive rotor is converted into a translational movement of the lancet and/or a lancet holder.

Such rotor drives have long been known. For example, a connecting rod for converting rotational movement to translational movement is disclosed in U.S. Pat. No. 4,924,879. In the disclosed embodiment, transverse forces are transferred via the connecting rod to the lancet. The transverse forces have a negative influence on the guidance of the lancet. In addition, the size of the wound formed in a body part is increased, which is perceived as painful by the patient.

With other rotor drives, a recess is provided in the drive rotor. The recess acts as a control cam being traced by a pin during the rotation. This control cam design allows very different relationships between the rotational movement and the translational movement, depending on the shape of the control cam. For example, a different control cam can be traced during the puncturing movement in the puncturing direction than during the retraction phase of the puncturing movement. Such a cam control is known from EP 1034740 A1, for example, in which a rotor which rotates about the longitudinal axis of the blood withdrawal device is provided.

EP 1504718 A2 also describes a blood withdrawal system having a drive rotor which rotates about an axis running perpendicular to the longitudinal direction of the system. Here again, a control cam is formed by a recess in which a control pin engages.

In known blood withdrawal devices, the control pins must be formed to be small in some cases due to the geometry, so there is the risk that the pin might break. These known devices also have in common the fact that the drives are force-guided, i.e., the coupling between the rotor and the lancet is such that each position of the rotor unambiguously corresponds to a position of the lancet.

Despite extensive development work in this field and the significant improvements thereby achieved, there is a great interest in a blood withdrawal system in which the creation of the wound is associated with the least possible pain, while the system is as simple as possible to operate, has a compact design and nevertheless has a certain sturdiness and is also inexpensive to manufacture.

### SUMMARY OF THE INVENTION

A blood withdrawal system according to one embodiment of the present invention comprises a housing and a lancet drive for driving a puncturing movement of a lancet along a predetermined puncturing path. The lancet drive has a drive spring and a drive rotor. The drive rotor is driven by the drive spring and rotates about an axis during the puncturing movement. The lancet drive also includes a coupling mechanism for converting the rotational movement of the drive rotor into the puncturing movement of the lancet. In a forward phase of the puncturing movement, the lancet is moved in the puncturing direction until its tip penetrates into the body part to create a wound. The coupling mechanism also moves the lancet in a retraction phase of the puncturing movement, which follows the forward phase, with the lancet being withdrawn from the skin during the retraction phase.

The coupling mechanism includes a translation element coupled to the lancet which has two guide walls each with a guide surface. The translation element is guided by a guide on a translational movement path. The coupling between the translation element and the lancet is formed so that a movement of the translation element on the translational movement path in a first direction produces a movement of the lancet in the forward phase of the puncturing direction. Likewise, the movement of the translation element in a second direction produces a movement of the lancet in the retraction phase in the direction opposite the puncturing direction. The two guide walls of the translation element each have a contact surface, with one contact surface being oriented in the first direction of the translation element and the other contact surface being oriented in the second direction of the movement of the translation element.

The orientation of the contact surface in the direction of the movement of the translation element is such that the surface normal of the contact surface has a component in the direction of movement of the translation element. The contact surface thus runs across the direction of movement of the translation element. The contact surface extends perpendicular to the direction of movement, so that the surface normal has only one component in the direction of movement of the translation element.

The operative connection between the movement of the translation element and the movement of the lancet can be accomplished through any suitable coupling. The translation element can be coupled directly to the lancet. The coupling can comprise, for example, an angle lever or a deflection to transmit the direction of movement of the translation element in a predetermined direction of the lancet movement.

The drive rotor of the blood withdrawal system has a control element with a control surface which runs around a center of the control element and is oriented radially outward from the center. During the puncturing movement, the control element rotates together with the drive rotor. The axis of rotation runs at a distance from the center of the control element. The control element is driven together with the drive rotor by the drive spring. The control surface of the control element is in contact with the guide surfaces on the guide walls of the translation element during the rotation of the drive rotor such that the movement of the translation element is controlled by the control element at least during a portion of the forward phase of movement and at least during a portion of the retraction phase of the puncturing movement.

A very direct conversion of the rotational movement of the drive rotor into a translational movement of the translation element can be implemented in this manner. This permits good guidance, so that suitable wounds are created in the patient's skin. In addition, very rapid puncturing of the body part can be implemented, resulting in a reduced pain perception by the patient. This type of coupling mechanism generally requires very few components, and accordingly, the blood withdrawal system may be manufactured inexpensively. Furthermore, complex mechanisms can be omitted, such as those required when using control cams.

The control surface of the control element is preferably a lateral surface in the sense that it is formed by a generating line which runs parallel to the axis of rotation of the drive rotor. However, the generating line need not necessarily be a straight line. If the generating line is curved, then it may be curved in such a manner that the resulting control surface has an outward convex curvature. It may run parallel to the axis of rotation along a line of its maximum distance from the center of the control element.

In embodiments, since the control element rotates about an axis of rotation spaced apart from the center of the control element, the control element thus executes an eccentric rotation in which its own center is different from the axis of rotation.

The guide walls of the translation element are oriented in such a manner that the first contact surface is oriented in the puncturing direction while the other contact surface of the second guide wall is oriented in a direction opposite the puncturing direction. This arrangement yields a direct interaction between the translation element and the lancet. The direction of movement of the translation element then corresponds to the direction of movement of the lancet. The contact surface is oriented in or against the puncturing direction. The surface normal of the contact surface thus has a component which is oriented in the direction of puncturing or in the direction opposite the puncturing direction. The surface may be oriented perpendicular to the puncturing direction.

The control surface of the control element is in contact with the guide surfaces of the translation element and therefore controls the movement of the translation element at least partially during the forward phase and the retraction phase, so at least a partial coupling is implemented. The lancet is thus guided directly by the control element via the coupling mechanism in parts of the forward phase and of the retraction phase of movement. This partial coupling can also be extended to the entire puncturing movement. In this case, the lancet is force-guided by the drive rotor. The result is direct coupling. Each position of the control element corresponds to a position of the translation element and thus ultimately to a position of the lancet. This coupling may be generally referred to as a synchronous coupling because the position of the control element can be allocated to a defined position of the lancet.

In embodiments of the invention, the control element can be formed in such a manner that its axis of rotation is either inside the control element or as an alternative, outside of the control element. If the axis of rotation is arranged inside the control element, then the control element can preferably be formed by a circumferential wall of the drive rotor. In embodiments of the invention, the control element may be part of the drive rotor.

If the axis of rotation is outside of the control element, and in rotation of the drive rotor the control element is turning on a circular path about the axis of rotation, the control element can still be a part of the drive rotor. In particular, the control element can be formed by a pin mounted on the drive rotor. It should be noted that the drive rotor need not necessarily be in the form of a disk or wheel. The geometry of the drive motor is not limited to a cam-type design. For example, the pin-shaped control element of the drive motor may be mounted on the end of a rotating arm.

In embodiments of the blood withdrawal system, at least the contact surfaces of the translation element are made from friction-optimized materials that are in contact with the control element. The entire translation element may be made of friction-optimized materials. It is also possible to provide the contact surfaces or the entire translation element with a coating having friction-optimized properties, such as all materials that are mixed with Polytetrafluoroethylene (PTFE) including Polyoxymethylene (POM) PTFE, for example. This reduces friction between the control element and the translation element. Wear is also reduced. On the whole, this yields simple handling of the device because the rotational movement of the control element is converted into the translational movement of the translation element without any significant friction losses.

The guidance of the translation element during its translational movement is formed by guide rails which may be arranged in the housing of the blood withdrawal system. Alternatively or additionally, a cylinder guide can also be used. In embodiments, the blood withdrawal system may be in the form of an elongated cylinder. The housing of the blood withdrawal system can serve as a guide for the translation element. Other types of guidance are also conceivable as long as they fulfill the purpose of ensuring the straightest possible puncturing path of the lancet and of avoiding any vibration that might occur.

### BRIEF DESCRIPTION OF THE DRAWINGS

The above-mentioned and other features of this invention and the manner of obtaining them will become more apparent and the invention itself will be better understood by reference to the following description of embodiments of the present invention taken in conjunction with the accompanying drawings, wherein:

FIG. 1 shows a cross-sectional view of a first embodiment of a blood withdrawal system;

FIG. 2 shows a cross-sectional view of a basic diagram of a second embodiment of a blood withdrawal system, and

FIG. 3 shows a detailed view of a coupling mechanism of the blood withdrawal system according to FIG. 2.

Corresponding reference characters indicate corresponding parts throughout the several views. Although the drawings represent embodiments of the present invention, the drawings are not necessarily to scale and certain features may be exaggerated in order to better illustrate and explain the present invention. The exemplifications set out herein illustrate embodiments of the invention, and such exemplifications are not to be construed as limiting the scope of the invention in any manner.

### DETAILED DESCRIPTION

For the purposes of promoting an understanding of the principles of the invention, reference will now be made to the embodiments illustrated in the drawings, which are described below. It will nevertheless be understood that no limitation of the scope of the invention is thereby intended. The invention includes any alterations and further modifications in the illustrated devices and described methods and further applications of the principles of the invention, which would normally occur to one skilled in the art to which the invention relates.

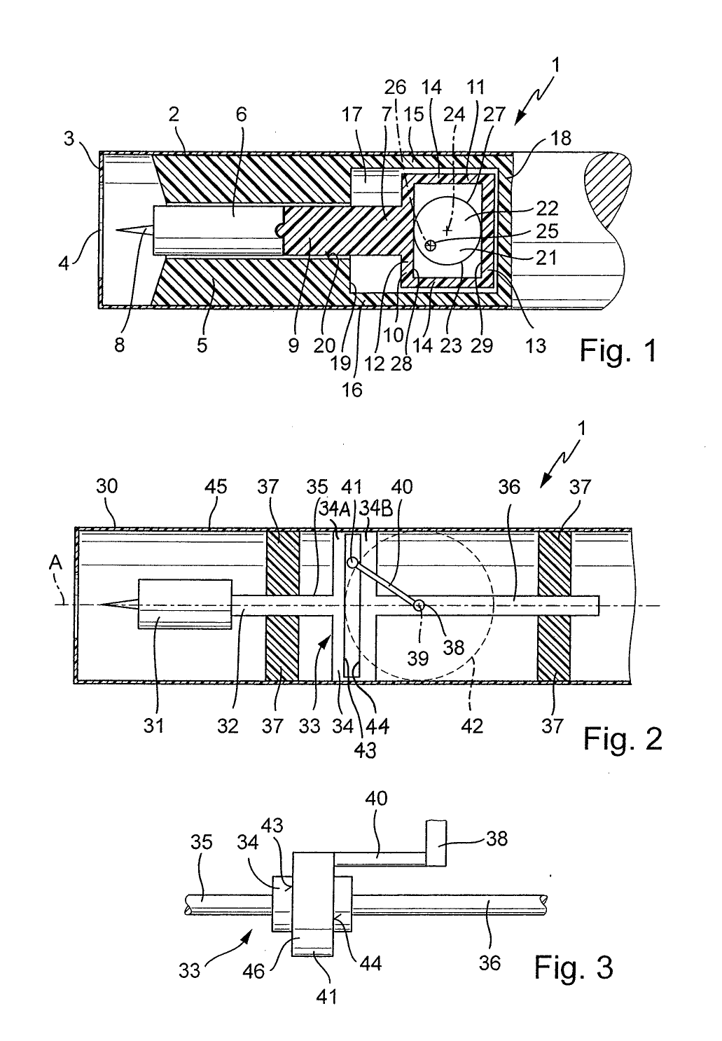

The blood withdrawal system 1 shown in FIG. 1 in the form of a basic schematic diagram has an elongated housing 2 with an opening 4 provided on its distal end 3. A lancet guide 5 is provided in the interior of the housing 2, guiding a lancet 6 on its puncturing path. The lancet 6 may also be guided by the housing 2 on its puncturing path, with the inside wall of the housing 2 functioning as the lancet guide 5.

The lancet 6 is coupled to a coupling mechanism 7 and is held by it in such a manner that the tip 8 of the lancet 6 is in the same position even if a new lancet 6 is coupled to the coupling mechanism 7. The coupling mechanism 7 includes a lancet holder 9, being adapted to the lancet 6. In the depicted embodiment, a translation element 10 in the form of a cage 11 is connected to the lancet holder 9 of the coupling mechanism 7. The cage 11 has a distal guide wall 12 and a proximal guide wall 13 as well as two side walls 14.

The translation element 10 of the coupling mechanism 7 is guided by a guide 15 connected to the housing 2 on a linear movement path. The guide 15 is formed as a cylinder guide 16 in the depicted embodiment and cylinder guide 16 guides the translation element 10 during the puncturing movement of the lancet 6. The guide 15 can be formed by the housing 2. In particular in the case of cylindrically formed housings 2, the cylinder guide 16 may already be formed by the inside wall of the housing 2. The guide 15 can also be implemented by the same component as the lancet guide 5, e.g., the lancet guide 5 can simultaneously function as the guide 15. In particular, when the housing 2 forms the lancet guide 5, housing 2 can simultaneously serve as a guide for the translation element 10.

The cylinder guide 16 illustrated in FIG. 1 forms a guide space 17 in the interior, which is bordered by a proximal wall 18. In the direction of the distal end 3, the guide space 17 is closed by a distal wall 19 having a bore 20. The lancet holder 9 of the coupling mechanism 7, being connected in one piece to the translation element 10, passes through the bore 20. The proximal wall 18 and the distal wall 19 of the guide space 17 are spaced apart from one another in such a manner that they do not restrict the path of movement of the translation element 10.

There is a drive rotor 21 inside the cage 11 of the translation element 10. The drive rotor 21 belongs to a lancet drive, which also includes parts not shown here, in particular a drive spring and a tension device for applying tension to the drive springs. During the puncturing movement of the lancet 6 the drive rotor 21 is driven by the drive spring and rotates about an axis 26.

A circumferential wall 23 of the drive rotor 21 oriented radially outward forms a control surface 27 in the embodiment shown here. In the embodiment according to FIG. 1, the drive rotor 21 thus also forms the control element 22. Its center 24, corresponding to the center of the control surface 27, is spaced apart from the axis of rotation 26. During rotation, the center 24 of the control element 22 runs on a circular path around the axis of rotation 26.

The control element 22 has a control surface 27 oriented radially outward from its center 24. The control surface 27 is formed such that the surface normals are directed radially outward from the center 24 along a line revolving about the center 24.

The control surface 27 of the control element 22 is in contact with a contact surface 28 of the distal guide wall 12 of the translation element 10 and with a contact surface 29 of the proximal guide wall 13 of the translation element 10. The contact surface 28 is oriented against the puncturing direction of the lancet 6, while the contact surface 29 is oriented in the puncturing direction.

FIG. 1 shows a permanent coupling of the control element 22 to the translation element 10 in which the control element 22 is substantially in contact with both contact surfaces 28 and 29 at the same time. In embodiments, the control element 22 is arranged to reduce the amount of friction between the element 22 and the surfaces 28 and 29. Such a coupling may be referred to as a forced coupling. The lancet 6 is force-guided, i.e., each position of the rotating control element 22 correlates with an unambiguous position of the translation element 10 and/or the lancet 6.

FIG. 1 shows the position of the control element 22 at the point of reversal of the puncturing movement. During the subsequent retraction phase of the puncturing movement, the control element 22 exerts a force on the contact surface 29 and forces the translation element 10 against the puncturing direction of the lancet 6. During the forward phase, the control element 22 acts with a force on the contact surface 28 and moves the translation element 10 in the puncturing direction. The rotational movement of the control element 22 is thus easily converted into a translational movement of the translation element 10, wherein a force of the control surface 27 acts on at least one of the contact surfaces 28, 29 during the rotation of the control element 22 about the axis of rotation 26.

The contact surfaces 28 and 29 of the translation element 10 preferably run in a plane perpendicular to the axis of rotation 26. Therefore, forces acting across the puncturing direction are prevented.

There are no transverse forces, so the total force exerted by the control element 22 is directed in or against the puncturing direction.

In one embodiment, at least the contact surface 28 of the translation element 10 is directed substantially perpendicular to the puncturing direction of the lancet 6. Both contact surfaces 28 and 29 may be directed perpendicular to the lancet 6. Therefore, the friction between the control element 22 and the translation element 10 is reduced and tilting of the translation element 10 on its translational path in the puncturing direction and against the puncturing direction is inhibited. A good transfer of force is supported by the guide 15, so that a vibration-free puncturing of the lancet 6 into the body part is achieved on the whole.

As an alternative to the translation element 10 shown in FIG. 1, the distance between the distal guide wall 12 and the proximal guide wall 13 can be greater than the diameter of the control element 22. The distance between the two guide walls 12 and 13 is selected by taking into account the diameter and eccentricity of the rotor, so that the control element 22 is in contact with one of the two contact surfaces 28, 29 of the translation element 10 during at least half of the puncturing movement, such that each position of the control element 22 correlates with a position of the translation element 10 and thus of the lancet 6. During the forward phase, the force of the control element 22 is transferred at least temporarily to the contact surface 28, and during the retraction phase, the force exerted by the control element 22 is transferred at least temporarily to the contact surface 29. In this embodiment, there is not a continuous force-guided coupling between the control element 22 and the translation element 10.

As an alternative to the translation element 10, which is formed as a cage 11, a translation element 10 comprising a distal guide wall 12 and a proximal guide wall 13 without connecting walls is also possible. These guide walls 12, 13 can be formed as webs, for example, which protrude away from a base body of the translation element. The control element 22 need not be surrounded. The contact between the control element 22 and the distal guide wall 12 and/or the proximal guide wall 13 must be ensured for only a portion of the forward phase and a portion of the retraction phase, so that at least temporary coupling between the control element 22 and the translation element 10 is implemented.

FIG. 2 shows an alternative embodiment of a blood withdrawal system 1, which has a housing 30 with a lancet guide and a lancet drive (not shown). A lancet 31 is connected to a coupling mechanism 32, comprising a translation element 33 which has a cage 34 with guide walls 34A and 34B and two opposing guide rods 35, 36. The guide rods 35, 36 extend along a central housing axis A, wherein the guide rod 35 is arranged between the cage 34 and the lancet 31. The guide rod 36 extends from the cage 34 away from the lancet 31. The rods 35, 36 are guided in the puncturing direction by a guide consisting of guide elements 37.

A drive rotor 38 is also arranged on the housing central axis A and may be rotated by a drive spring (not shown) of the lancet drive during the puncturing movement of the lancet 31. The drive rotor 38 rotates about an axis of rotation 39. A rotor arm 40, which is attached to the drive rotor 38, can also be in one piece with the drive rotor 38, as shown here. A control element 41 which is surrounded by the cage 34 is fixedly coupled to the distal end of the rotor arm 40.

In the exemplary embodiment illustrated in FIG. 2, the axis of rotation 39 is outside of the control element 41 and the control element 41 rotates on a circular path 42 (shown with a dotted line) which describes a circle about the axis of rotation 39 in the rotation of the drive rotor 38.

The control element 41 is always connected to the cage 34 in its rotational movement about the axis of rotation 39. The control element 41 is thus in contact with the contact surfaces 43, 44 of the cage 34, so that there is a direct coupling between the drive rotor 38 and the translation element 33 and thus ultimately with the lancet 31.

The cage 34 itself is also guided in the housing 30, where the inside walls of the housing 30 are formed as guide rails 45. Transverse forces are minimized in particular due to the embodiment of the translation element 33 as a cage 34 with the contact surfaces 43, 44 perpendicular to the puncturing direction. The transfer of force in the puncturing direction and in the direction opposite the puncturing direction is optimized.

FIG. 3 shows a longitudinal section along the central axis A of the housing from FIG. 2. The drive rotor 38, which consists of only a bearing shaft, is fixed to the rotor arm 40. A drive mechanism (not shown) is arranged on the end (not shown) of the drive rotor 38 in the form of a shaft (not shown). At its other end, the rotor arm 40 is connected to the control element 41, which is preferably formed as a pin 46. Due to the rigid coupling via the rotor arm 40, there is a fixed connection between the pin 46 and the drive rotor 38. The pin 46 transmits the forces exerted by the drive rotor 38 to the contact surfaces 43 and 44 of the cage 34 of the translation element 33.

It can be seen clearly in FIG. 3 that the pin 46 cannot slip out of the cage 34 because it protrudes beyond the cage 34 and there is too little play between the pin 46 and the straight contact surfaces 43, 44 of the cage 34. In particular, the pin 46 is prevented from slipping out if the guide is formed so that the translation element 33 can execute only a translational movement into and against the puncturing direction of the lancet 31, and if a movement in the two other directions in space, in particular a tilting, is excluded.

The coupling of the translation element 32 and the lancet 31 is such that a movement of the translation element 32 in a first direction causes a movement of the lancet 31 in the forward phase in the puncturing direction. A movement of the translation element 32 in a second direction causes a movement of the lancet 31 in the retraction phase opposite the puncturing direction. The guide surfaces 43, 44 are oriented so that the one guide surface is oriented in the first direction of movement of the translation element 32 and the other guide surface is oriented in the second direction of movement of the translation element 32.

While the invention has been taught with specific reference to these embodiments, one skilled in the art will recognize that changes can be made in form and detail without departing from the spirit and scope of the invention. The described embodiments are to be considered, therefore, in all respects only as illustrative and not restrictive. As such, the scope of the invention is indicated by the following claims rather than by the description.

1. A blood withdrawal system for producing blood from a body part for diagnostic purposes, comprising:
a housing;
a drive rotor capable of rotating about an axis, the drive rotor including a control element including a center and a control surface encompassing the center, the control element orientated radially outward from the center; and
a coupling mechanism including a translation element having at least two guide walls each including a contact surface, the translation element coupled to a lancet and guided by a guide on a translational movement path;
wherein during a puncturing movement, the control element rotates with the drive rotor about an axis of rotation spaced apart from the center of the control element and the control surface of the control element is in contact with the contact surfaces of at least one of the guide walls of the translation element during the rotation of the drive rotor such that the movement of the translation element is controlled by the drive rotor at least during a portion of the forward phase and at least during a portion of the retraction phase of the puncturing movement.

2. The blood withdrawal system according to claim 1 wherein the axis of rotation extends through a boundary defined by the control surface of the control element.

3. The blood withdrawal system according to claim 1 wherein the control element is formed by a circumferential wall of the drive rotor.

4. The blood withdrawal system according to claim 1 wherein the axis of rotation is located outside of the control surface of the control element.

5. The blood withdrawal system according to claim 1 wherein the control element revolves during rotation of the drive rotor on a circular path about the axis of rotation.

6. The blood withdrawal system according to claim 1 wherein the control element is a pin.

7. The blood withdrawal system according to claim 1 wherein the control element is in contact with the two contact surfaces of the guide walls of the translational element during at least half of the puncturing movement so that each position of the control element corresponds to the position of the translation element and the lancet.

8. The blood withdrawal system according to claim 1 wherein one contact surface of one of the guide walls of the translation element is oriented in the direction of the puncturing movement and another contact surface of another of the guide walls of the translation element is oriented in the direction opposite the puncturing direction.

9. The blood withdrawal system according to claim 1 wherein the contact surfaces of the guide walls of the translation element are located in a plane perpendicular to the axis of rotation.

10. The blood withdrawal system according to claim 1 wherein the contact surfaces are oriented essentially perpendicular to the puncturing direction of the lancet.

11. The blood withdrawal system according to claim 1 wherein at least the contact surfaces of the translation element that contact the control element are manufactured from a material including polytetrafluoroethylene.

12. The blood withdrawal system according to claim 1 wherein the guide comprises a plurality of guide rails.

13. The blood withdrawal system according to claim 1 wherein the guide comprises a guide cylinder.

14. The blood withdrawal system according to claim 1 wherein the translation element is a cage encompassing at least a portion of the control element.

15. The blood withdrawal system according to claim 1 wherein the translational element is coupled to the lancet in a manner allowing the translational element to propel the lancet in the puncturing direction.

16. The blood withdrawal system according to claim 14 wherein the translational element is coupled to the lancet in a manner allowing the translational element to move the lancet in a direction substantially opposite the puncturing direction.

17. A blood withdrawal system including:
a housing including an opening;
a lancet at least partially located in the housing and capable of extending at least partially through the opening;
a mechanism coupled to the lancet in a manner allowing the mechanism to drive the lancet in at least two directions, the mechanism including a first wall and a second wall spaced apart from the first wall defining a gap; and
an assembly including a surface at least partially located within the gap, the surface configured to contact at least one of the walls of the mechanism;
wherein the surface exerts a force against one of the walls in order to drive the lancet in a first direction.

18. The blood withdrawal system as set forth in claim 17 wherein the assembly further includes a rotor and the surface is connected to the rotor.

19. The blood withdrawal system as set forth in claim 18 wherein the surface is located on the rotor.

20. The blood withdrawal system as set forth in claim 17 wherein the rotor is at least partially located within the gap.

21. The blood withdrawal system as set forth in claim 17 wherein the rotor contacts both the first wall of the mechanism and the second wall of the mechanism.

22. The blood withdrawal system as set forth in claim 17 wherein the rotor includes a center and a rotation axis, and the center is spaced apart from the axis.

23. The blood withdrawal system as set forth in claim 17 wherein rotation of the rotor in a first direction propels the lancet in a first direction and rotation of the rotor in a second direction propels the lancet in a second direction opposite the first direction.

24. The blood withdrawal system as set forth in claim 17 wherein the mechanism includes a pair of side walls connecting the first wall to the second wall.

25. The blood withdrawal system as set forth in claim 17 wherein the assembly further includes an arm and an element comprising the surface, the arm connecting the element to the rotor.

26. The blood withdrawal system as set forth in claim 25 wherein the element is at least partially located within the gap.

27. The blood withdrawal system as set forth in claim 26 wherein the rotor includes a center about which it rotates and rotation of the rotor in a first direction results in the lancet moving in a first direction.

28. The blood withdrawal system as set forth in claim 26 wherein the rotor can rotate between a first position and a second position and the rotation of the rotor from the first position to the second position results in the lancet traveling in a first direction until the rotor reaches a midpoint of rotation and the lancet traveling in a second direction until the rotor reaches the second position.

29. The blood withdrawal system as set forth in claim 25 wherein the element is a pin.

30. The blood withdrawal system as set forth in claim 17 wherein the mechanism further includes a guide member and a guide element including an aperture and the guide member extends through the aperture of the guide element.

31. The blood withdrawal system as set forth in claim 30 wherein the mechanism further includes a second guide member and a second guide element including an aperture and the second guide member extends through the aperture of the second guide element.

32. The blood withdrawal system as set forth on claim 31 wherein the assembly is at least partially located between the guide elements.

33. The blood withdrawal system as set forth in claim 17 wherein the surface is in contact with the first wall and the second wall.
